(19) United States
(12) Patent Application Publication
(10) Pub. No.: US 2023/0285680 A1
(43) Pub. Date: Sep. 14, 2023

(54) DRIVING MECHANISM FOR DRIVING A PLUNGER OF AN AUTO-INJECTOR TO SLIDE RELATIVE TO A RESERVOIR OF THE AUTO-INJECTOR AND AUTO-INJECTOR THEREWITH

(71) Applicant: ALTEK BIOTECHNOLOGY CORPORATION, Hsinchu City (TW)

(72) Inventor: Yu-Cheng Huang, Hsinchu City (TW)

(73) Assignee: ALTEK BIOTECHNOLOGY CORPORATION, Hsinchu City (TW)

(21) Appl. No.: 17/844,038

(22) Filed: Jun. 19, 2022

Related U.S. Application Data
(60) Provisional application No. 63/318,785, filed on Mar. 11, 2022.

(51) Int. Cl. A61M 5/315; A61M 5/20

(52) U.S. Cl. CPC A61M 5/31528; A61M 5/20; A61M 2005/31588

(57) ABSTRACT

A driving mechanism for driving a plunger of an auto-injector to slide relative to a reservoir of the auto-injector is provided. The driving mechanism includes a first transmission component, a driving component, a second transmission component, a third transmission component, a sliding component and a bracket. The driving component is for driving the first transmission component to rotate to drive the second transmission component to rotatably drive the third transmission component rotate together with the second transmission component, so as to drive the sliding component to slide relative to the third transmission component by the third transmission component to push the plunger to pump a drug out of the reservoir. The sliding component passes through a guiding portion of the bracket, and the guiding portion is configured to guide the sliding component to slide without any rotation. Besides, a related auto-injector is provided.

### CROSS REFERENCE TO RELATED APPLICATIONS

This application claims the benefit of U.S. Provisional Application No. 63/318,785, filed on Mar. 11, 2022. The content of the application is incorporated herein by reference.

### BACKGROUND OF THE INVENTION

### 1. Field of the Invention

The present invention relates to a driving mechanism and an auto-injector therewith, and more specifically, to a driving mechanism for driving a plunger of an auto-injector to slide relative to a reservoir of the auto-injector and an auto-injector therewith.

### 2. Description of the Prior Art

An auto-injector, e.g., an on-body injector, is a medical device designed to deliver a dose of a drug. However, the conventional auto-injectors available in the markets are unable to meet requirements of small volume, high driving power, long driving distance, long injecting period and accurate drug dose delivery rate. Therefore, an improvement of the auto-injector is urgently needed.

### SUMMARY OF THE INVENTION

It is an objective of the present invention to provide a driving mechanism for driving a plunger of an auto-injector to slide relative to a reservoir of the auto-injector and an auto-injector therewith for solving the aforementioned problem.

In order to achieve the aforementioned objective, the present invention discloses a driving mechanism for driving a plunger of an auto-injector to slide relative to a reservoir of the auto-injector. The driving mechanism includes a first transmission component, a driving component, a second transmission component, a third transmission component, a sliding component and a bracket. The driving component is coupled to the first transmission component and for driving the first transmission component to rotate. The second transmission component is rotatably engaged with the first transmission component. The second transmission component is driven by the first transmission component to rotate when the driving component drives the first transmission component to rotate. The third transmission component is fixedly connected to the second transmission component. The third transmission component is driven by the second transmission component to rotate when the first transmission component drives the second transmission component to rotate. The sliding component is at least partially slidably disposed inside the third transmission component and coupled to the third transmission component. The sliding component is connected to the plunger. The sliding component is driven by the third transmission component to slide relative to the third transmission component when the third transmission component rotates. The bracket includes a guiding portion. The sliding component passes through the guiding portion. The third transmission component is rotatable relative to the bracket, and the guiding portion is configured to guide the sliding component to slide without a rotation.

According to an embodiment of the present invention, when the driving component drives the first transmission component to rotate around a first rotating axis, the first transmission component drives the second transmission component to rotate around a second rotating axis so as to drive the third transmission component to rotate around the second rotating axis together with the second transmission component, so that the sliding component is driven to slide relative to the third transmission component along a sliding direction parallel to the second rotating axis without the rotation around the second rotating axis.

According to an embodiment of the present invention, the first transmission component is a worm screw. The second transmission component is a worm gear. The third transmission component is a screw sleeve. The sliding component is a screw rod, and the driving component is an electric motor.

According to an embodiment of the present invention, the driving mechanism further includes a reducer coupled between the driving component and the first transmission component.

According to an embodiment of the present invention, the reducer is a gearbox.

According to an embodiment of the present invention, the guiding portion includes a sliding through hole structure. The sliding component slidably passes through the sliding through hole structure. A cross section of the sliding component matches with a cross section of the sliding through hole structure. The sliding component includes at least one first arc part and at least one first flat part connected to the at least one first arc part. The sliding through hole structure includes at least one second arc part and at least one second flat part connected to the at least one second arc part, and the at least one second arc part and the at least one second flat part are respectively corresponding to the at least one first arc part and the at least one first flat part.

According to an embodiment of the present invention, an internal thread structure is formed on an inner periphery of the third transmission component, and an outer thread structure is formed on the at least one first arc part of the sliding component.

According to an embodiment of the present invention, the bracket further includes a holding portion. The holding portion includes a rotation through hole structure, and the third transmission component rotatably passes through the rotation through hole structure.

According to an embodiment of the present invention, the bracket further includes a supporting portion. The supporting portion includes a platform structure, and the platform structure is configured to support a side of the third transmission component.

According to an embodiment of the present invention, the bracket further includes an accommodating portion. The accommodating portion includes an L-shaped structure, and the L-shaped structure is configured to accommodate the first transmission component.

In order to achieve the aforementioned objective, the present invention further discloses an auto-injector. The auto-injector includes a reservoir, a plunger and a driving mechanism. The plunger is slidably disposed inside the reservoir. The driving mechanism is for driving the plunger to slide relative to the reservoir. The driving mechanism includes a first transmission component, a driving component, a second transmission component, a third transmission component, a sliding component and a bracket. The driving component is coupled to the first transmission component and for driving the first transmission component to rotate. The second transmission component is rotatably engaged with the first transmission component. The second transmission component is driven by the first transmission component to rotate when the driving component drives the first transmission component to rotate. The third transmission component is fixedly connected to the second transmission component. The third transmission component is driven by the second transmission component to rotate when the first transmission component drives the second transmission component to rotate. The sliding component is at least partially slidably disposed inside the third transmission component and coupled to the third transmission component. The sliding component is connected to the plunger. The sliding component is driven by the third transmission component to slide relative to the third transmission component when the third transmission component rotates. The bracket includes a guiding portion. The sliding component passes through the guiding portion. The third transmission component is rotatable relative to the bracket, and the guiding portion is configured to guide the sliding component to slide without a rotation.

According to an embodiment of the present invention, when the driving component drives the first transmission component to rotate around a first rotating axis, the first transmission component drives the second transmission component to rotate around a second rotating axis so as to drive the third transmission component to rotate around the second rotating axis together with the second transmission component, so that the sliding component is driven to slide relative to the third transmission component along a sliding direction parallel to the second rotating axis without the rotation around the second rotating axis.

According to an embodiment of the present invention, the first transmission component is a worm screw. The second transmission component is a worm gear. The third transmission component is a screw sleeve. The sliding component is a screw rod, and the driving component is an electric motor.

According to an embodiment of the present invention, the driving mechanism further includes a reducer coupled between the driving component and the first transmission component.

According to an embodiment of the present invention, the reducer is a gearbox.

According to an embodiment of the present invention, the guiding portion includes a sliding through hole structure. The sliding component slidably passes through the sliding through hole structure. A cross section of the sliding component matches with a cross section of the sliding through hole structure. The sliding component includes at least one first arc part and at least one first flat part connected to the at least one first arc part. The sliding through hole structure includes at least one second arc part and at least one second flat part connected to the at least one second arc part, and the at least one second arc part and the at least one second flat part are respectively corresponding to the at least one first arc part and the at least one first flat part.

According to an embodiment of the present invention, an internal thread structure is formed on an inner periphery of the third transmission component, and an outer thread structure is formed on the at least one first arc part of the sliding component.

According to an embodiment of the present invention, the bracket further includes a holding portion. The holding portion includes a rotation through hole structure, and the third transmission component rotatably passes through the rotation through hole structure.

According to an embodiment of the present invention, the bracket further includes a supporting portion. The supporting portion includes a platform structure, and the platform structure is configured to support a side of the third transmission component.

According to an embodiment of the present invention, the bracket further includes an accommodating portion. The accommodating portion includes an L-shaped structure, and the L-shaped structure is configured to accommodate the first transmission component.

In summary, in the present invention, the driving mechanism not only has compact structure and high power and high efficiency transmission but also achieves a long sliding distance and a slow sliding speed of the sliding component and prevents any rotation of the sliding component during a sliding movement of the sliding component. Therefore, the present invention can meet requirements of small volume, high driving power, long driving distance, long injecting period and accurate drug dose delivery rate.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 and FIG. 2 are partial diagrams of an auto-injector at different views according to an embodiment of the present invention.

FIG. 3 and FIG. 4 are partial exploded diagrams of the auto-injector at different views according to the embodiment of the present invention.

FIG. 5 and FIG. 6 are partial diagrams of a driving mechanism according to the embodiment of the present invention.

FIG. 7 is a partial sectional diagram of the driving mechanism according to the embodiment of the present invention.

FIG. 8 and FIG. 9 are diagrams of the auto-injector in different states according to the embodiment of the present invention.

### DETAILED DESCRIPTION

In the following detailed description of the preferred embodiments, reference is made to the accompanying drawings which form a part hereof, and in which is shown by way of illustration specific embodiments in which the invention may be practiced. In this regard, directional terminology, such as “top”, “bottom”, “left”, “right”, “front”, “back”, etc., is used with reference to the orientation of the Figure(s) being described. The components of the present invention can be positioned in a number of different orientations. As such, the directional terminology is used for purposes of illustration and is in no way limiting. Accordingly, the drawings and descriptions will be regarded as illustrative in nature and not as restrictive. Also, if not specified, the term “connect” or “couple” is intended to mean either an indirect or direct electrical/mechanical connection. Thus, if a first device is connected or coupled to a second device, that connection may be through a direct electrical/mechanical connection, or through an indirect electrical/mechanical connection via other devices and connections.

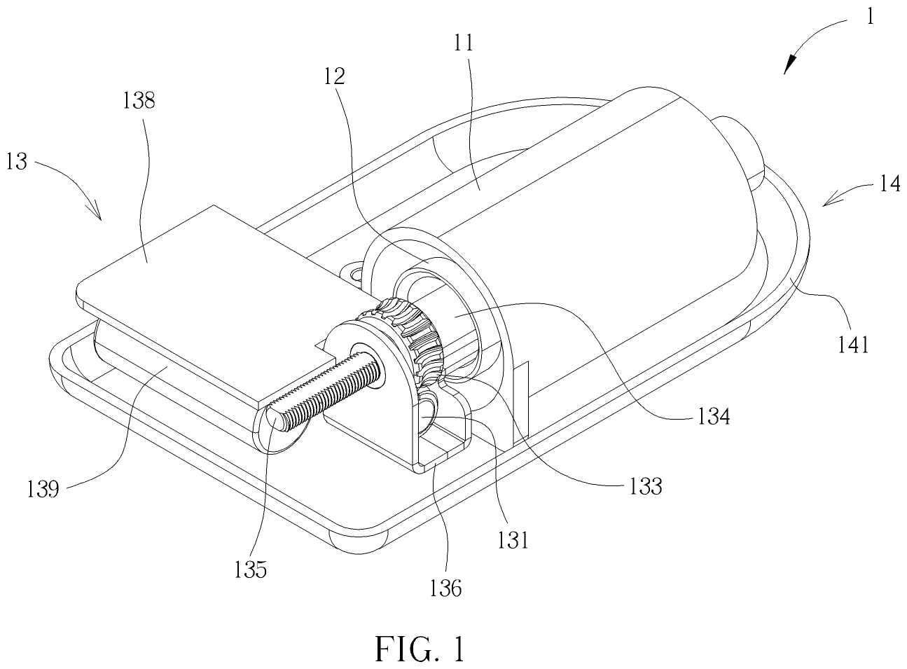

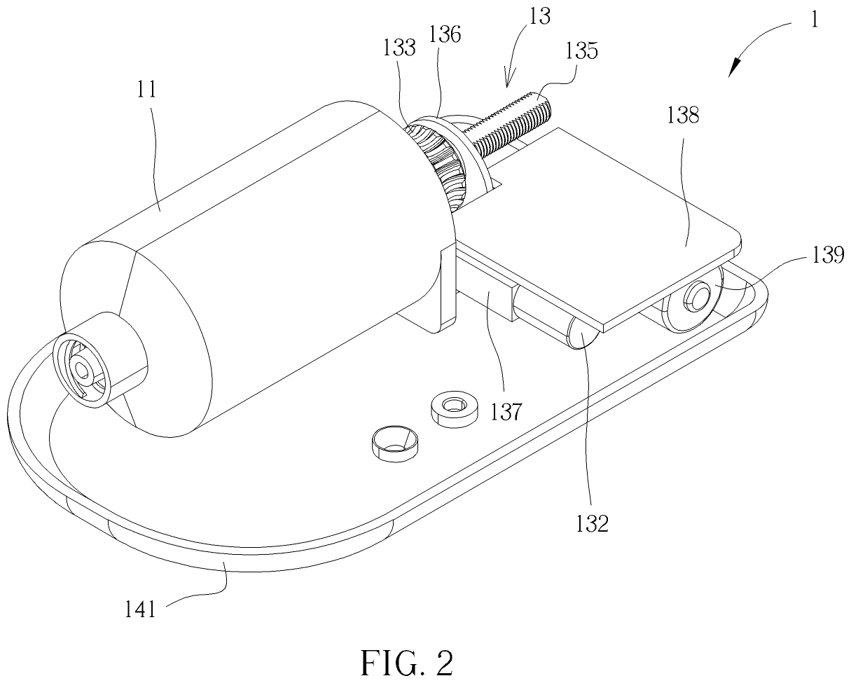

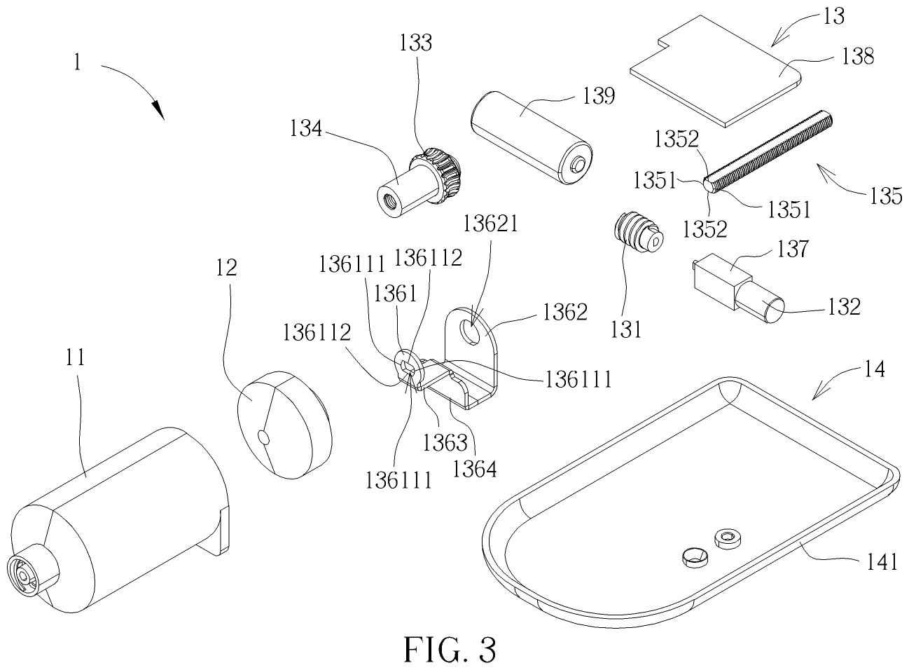

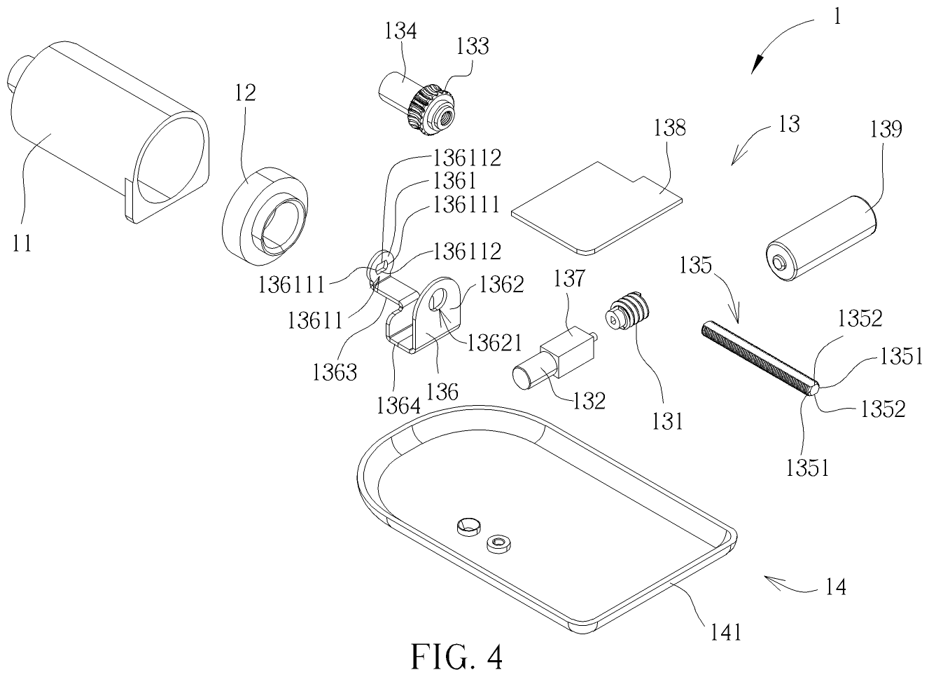

Please refer to FIG. 1 to FIG. 4. FIG. 1 and FIG. 2 are partial diagrams of an auto-injector 1 at different views according to an embodiment of the present invention. FIG. 3 and FIG. 4 are partial exploded diagrams of the auto-injector 1 at different views according to the embodiment of the present invention. As shown in FIG. 1 to FIG. 4, the auto-injector 1 includes a reservoir 11, a plunger 12 and a driving mechanism 13. The plunger 12 is slidably disposed inside the reservoir 11. The driving mechanism 13 is for driving the plunger 12 to slide relative to the reservoir 11.

In this embodiment, the auto-injector 1 further includes a case 14. The case 14 includes a first mounting part 141 and a second mounting part, which is not shown in the figures, detachable installed on the first mounting part 141. The reservoir 11 and the driving mechanism 13 are mounted on the first mounting part 141. The case 14 is configured to conceal the reservoir 11 and the driving mechanism. 13 for preventing damage of the reservoir 11 and the driving mechanism 13.

However, the present invention is not limited to this embodiment. For example, in another embodiment, the case can be a one-piece structure with an opening to expose the reservoir and the driving mechanism.

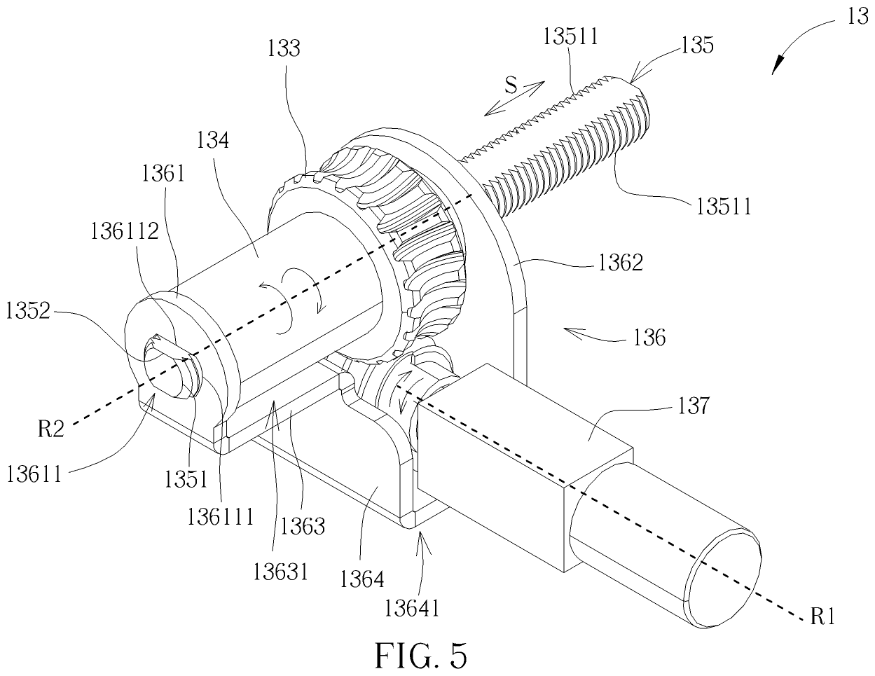

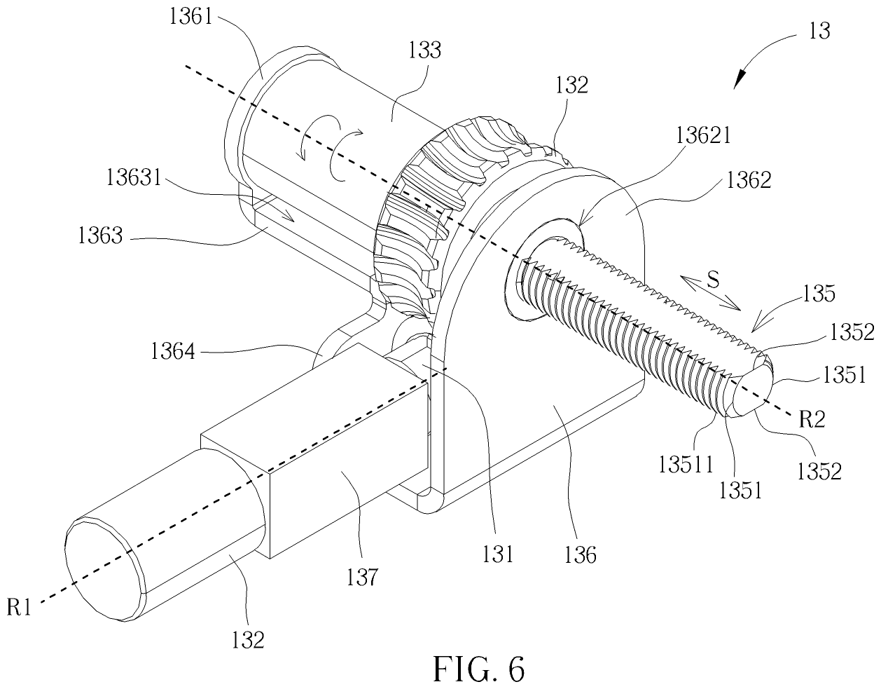

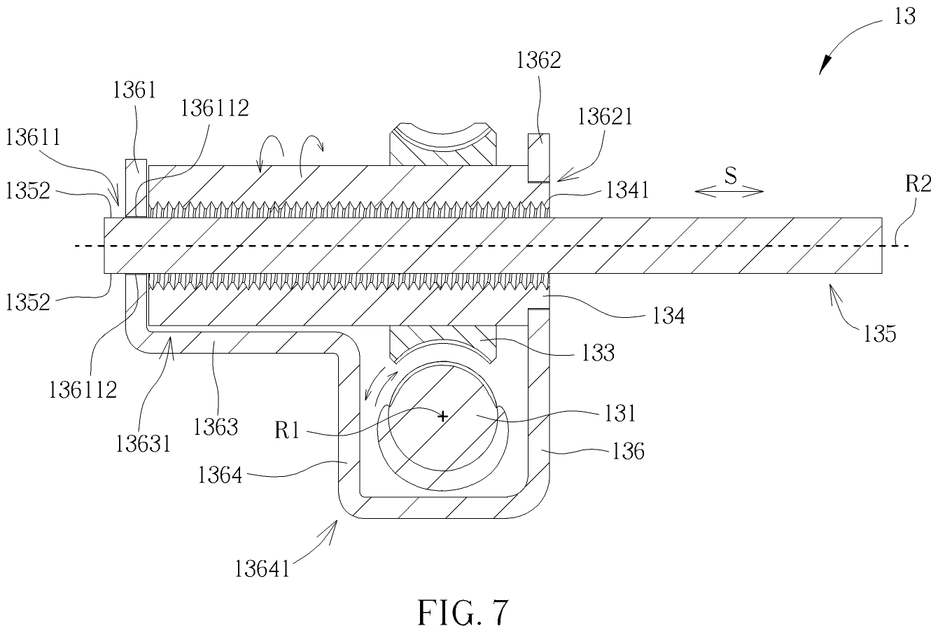

Please refer to FIG. 1 to FIG. 7. FIG. 5 and FIG. 6 are partial diagrams of the driving mechanism 13 according to the embodiment of the present invention. FIG. 7 is a partial sectional diagram of the driving mechanism 13 according to the embodiment of the present invention. As shown in FIG. 1 to FIG. 7, the driving mechanism 13 includes a first transmission component 131, a driving component 132, a second transmission component 133, a third transmission component 134, a sliding component 135 and a bracket 136. The driving component 132 is coupled to the first transmission component 131 and for driving the first transmission component 131 to rotate. The second transmission component 133 is rotatably engaged with the first transmission component 131. The second transmission component 133 is driven by the first transmission component 131 to rotate when the driving component 132 drives the first transmission component 131 to rotate. The third transmission component 134 is fixedly connected to the second transmission component 133, e.g., by a tightly fitting manner or an integrally forming manner. The third transmission component 134 is driven by the second transmission component 133 to rotate when the first transmission component 131 drives the second transmission component 133 to rotate. The sliding component 135 is at least partially slidably disposed inside the third transmission component 134 and coupled to the third transmission component 134. The sliding component 135 is connected to the plunger 12. The sliding component 135 is driven by the third transmission component 134 to slide relative to the third transmission component 134 when the third transmission component 134 rotates. The bracket 136 includes a guiding portion 1361. The sliding component 135 passes through the guiding portion 1361. The third transmission component 134 is rotatable relative to the bracket 136, and the guiding portion 1361 is configured to guide the sliding component 135 to slide without any rotation.

When the driving component 132 drives the first transmission component 131 to rotate around a first rotating axis R1, the first transmission component 131 drives the second transmission component 133 to rotate around a second rotating axis R2 perpendicular to the first rotating axis R1 so as to drive the third transmission component 134 to rotate around the second rotating axis R2 together with the second transmission component 133, so that the sliding component 135 is driven to slide relative to the third transmission component 134 along a sliding direction S parallel to the second rotating axis R2 without any rotation around the second rotating axis R2.

Specifically, the first transmission component 131 can be a worm screw. The second transmission component 133 can be a worm gear. The third transmission component 134 can be a screw sleeve. The sliding component 135 can be a screw rod. The driving component 132 can be an electric motor. However, the present invention is not limited to this embodiment. It depends on practical demands. For example, in another embodiment, the driving component can be a pneumatic motor, and the first transmission component can be a smaller gear wheel. The second transmission component can be a larger gear wheel.

Besides, in this embodiment, as shown in FIG. 1 to FIG. 7, the driving mechanism 13 further includes a reducer 137 coupled between the driving component 132 and the first transmission component 131. An input shaft and an output shaft of the reducer 137 can be respectively connected to the driving component 132 and the first transmission component 131. The reducer 137 can have various reduction ratios to control a rotating speed of the first transmission component 131, so as to control a sliding speed of the sliding component 135.

Specifically, the reducer 137 can be a gearbox. However, the present invention is not limited to this embodiment. It depends on practical demands. For example, in another embodiment, the reducer can be a pulley and belt system. Alternatively, in another embodiment, the reducer can be omitted.

It should be noticed that the aforementioned configuration can not only have small occupied space and achieve high power and high efficiency transmission but also allow a rotating speed of the third transmission component 134 to be adjusted by adjusting a first reduction ratio between the first transmission component 131 and the second transmission component 133 and/or a second reduction ratio between the driving component 132 and the first transmission component, e.g., a gear ratio of the reducer 137.

Furthermore, in this embodiment, as shown in FIG. 1 to FIG. 4, the driving mechanism 13 further includes a control unit 138 and a power source 139. The control unit 138 can be a circuit board for controlling the driving component 132 to actuate or stop a rotating movement of the first transmission component 131 and to control the rotating speed or a rotating direction of the first transmission component 131. The power source 139 can be a battery for providing electricity to the control unit 138 and the driving component 132.

In order to achieve configuration of the guiding portion 1361 to guide the sliding component 135 to slide without any rotation when the third transmission component 134 rotatably drives the sliding component 135 to slide, as shown in FIG. 3 to FIG. 7, the guiding portion 1361 includes a sliding through hole structure 13611. The sliding component 135 slidably passes through the sliding through hole structure 13611. A cross section of the sliding component 135 matches with a cross section of the sliding through hole structure 13611. The sliding component 135 includes two first arc parts 1351 opposite to each other, and two first flat parts 1352 opposite to each other and connected to the two first arc parts 1351. The sliding through hole structure 13611 includes two second arc parts 136111 opposite to each other, and two second flat parts 136112 opposite to each other and connected to the two second arc parts 136111. The two second arc parts 136111 and the two second flat parts 136112 are respectively corresponding to the two first arc parts 1351 and the two first flat parts 1352. An internal thread structure 1341 is formed on an inner periphery of the third transmission component 134, e.g., by plastic injection molding or insert molding, and an outer thread structure 13511 is formed on each of the first arc parts 1351 of the sliding component 135. The aforementioned configuration can ensure no rotation of the sliding component 135 during a sliding movement of the sliding component 135 by a cooperation of the sliding component 135 and the sliding through hole structure 13611, so as to meet a requirement of accurate drug dose delivery rate.

However, the structures of the sliding component and the guiding portion are not limited to this embodiment. For example, in another embodiment, the sliding component can include only one first arc part and one first flat part connected to the first arc part, and the sliding through hole structure can include only one second arc parts and one second flat part connected to the second arc part.

In addition, in order to achieve a stable rotating movement of the third transmission component 134 and make structure of the driving mechanism reasonably compact, as shown in FIG. 3 to FIG. 7, the bracket 136 further includes a holding portion 1362, a supporting portion 1363 and an accommodating portion 1364. The holding portion 1362 is opposite to the guiding portion 1361 and includes a rotation through hole structure 13621. The third transmission component 134 rotatably passes through the rotation through hole structure 1362 and is located between the holding portion 1362 and the guiding portion 1361. The supporting portion 1363 is connected to the guiding portion 1361 and includes a platform structure 13631. The platform structure 13631 can support a lower side of the third transmission component 134 to prevent the third transmission component 134 from accidently falling down. The accommodating portion 1364 is connected to the supporting portion 1363 and the holding portion 1362 and includes an L-shaped structure 13641 for accommodating the first transmission component 131.

However, the present invention is not limited to this embodiment. It depends on practical demands. For example, in another embodiment, the accommodating portion can include a bending structure, a step-shaped structure, or a U-shaped structure. Alternatively, in another embodiment, at least one of the holding portion, the supporting portion and the accommodating portion can be omitted.

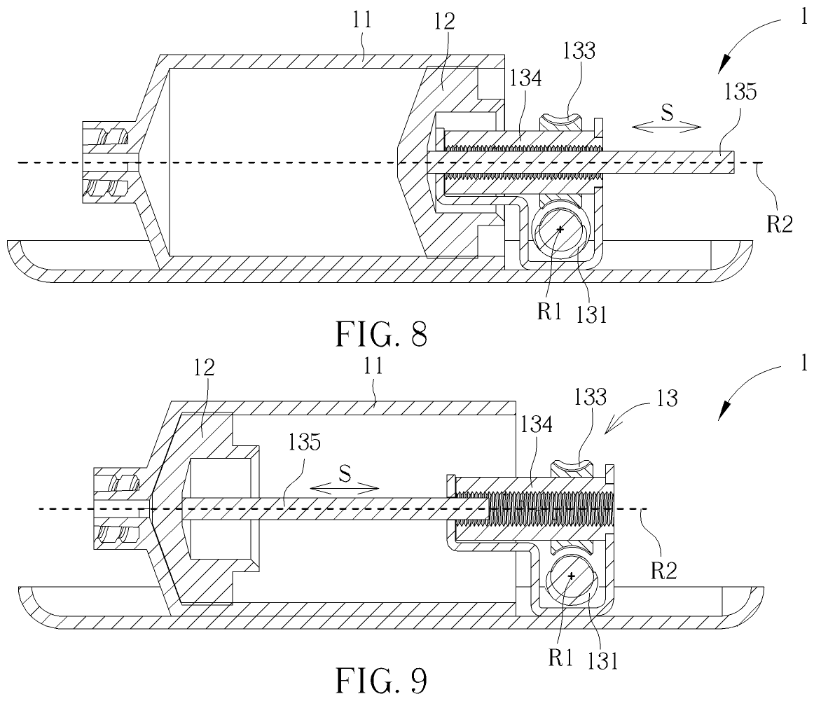

Detailed description for operational principle of the auto-injector 1 is provided as follows. Please further refer to FIG. 5 to FIG. 9. FIG. 8 and FIG. 9 are diagrams of the auto-injector 1 in different states according to the embodiment of the present invention. As shown in FIG. 5 to FIG. 9, after the auto-injector 1 is attached on a patient's body, the driving component 132 can be controlled by the control unit 138 to drive the first transmission component 131 to rotate around the first rotating axis R1 with the reducer 137. When the first transmission component 131 is driven to rotate around the first rotating axis R1, the first transmission component 131 drives the second transmission component 133 to rotate around the second rotating axis R2 so as to drive the third transmission component 134 to rotate around the second rotating axis R2 together with the second transmission component 133, so that the sliding component 135 can be driven to slide relative to the third transmission component 134 along the sliding direction S without any rotation, so as to drive the plunger 12 to slide relative to the reservoir 11 from a position as shown in FIG. 8 to a position as shown in FIG. 9 to stably pump a drug out of the reservoir 11 to complete a drug injection.

In contrast to the prior art, in the present invention, the driving mechanism not only has compact structure and high power and high efficiency transmission but also achieves a long sliding distance and a slow sliding speed of the sliding component and prevents any rotation of the sliding component during a sliding movement of the sliding component. Therefore, the present invention can meet requirements of small volume, high driving power, long driving distance, long injecting period and accurate drug dose delivery rate.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A driving mechanism for driving a plunger of an auto-injector to slide relative to a reservoir of the auto-injector, the driving mechanism comprising:
a first transmission component;
a driving component coupled to the first transmission component and for driving the first transmission component to rotate;
a second transmission component rotatably engaged with the first transmission component, the second transmission component being driven by the first transmission component to rotate when the driving component drives the first transmission component to rotate;
a third transmission component fixedly connected to the second transmission component, the third transmission component being driven by the second transmission component to rotate when the first transmission component drives the second transmission component to rotate;
a sliding component at least partially slidably disposed inside the third transmission component and coupled to the third transmission component, the sliding component being connected to the plunger, the sliding component being driven by the third transmission component to slide relative to the third transmission component when the third transmission component rotates; and
a bracket comprising a guiding portion, the sliding component passing through the guiding portion, the third transmission component being rotatable relative to the bracket, and the guiding portion being configured to guide the sliding component to slide without a rotation.

2. The driving mechanism of claim 1, wherein when the driving component drives the first transmission component to rotate around a first rotating axis, the first transmission component drives the second transmission component to rotate around a second rotating axis so as to drive the third transmission component to rotate around the second rotating axis together with the second transmission component, so that the sliding component is driven to slide relative to the third transmission component along a sliding direction parallel to the second rotating axis without the rotation around the second rotating axis.

3. The driving mechanism of claim 1, wherein the first transmission component is a worm screw, the second transmission component is a worm gear, the third transmission component is a screw sleeve, the sliding component is a screw rod, and the driving component is an electric motor.

4. The driving mechanism of claim 1, further comprising a reducer coupled between the driving component and the first transmission component.

5. The driving mechanism of claim 4, wherein the reducer is a gearbox.

6. The driving mechanism of claim 1, wherein the guiding portion comprises a sliding through hole structure, the sliding component slidably passes through the sliding through hole structure, a cross section of the sliding component matches with a cross section of the sliding through hole structure, the sliding component comprises at least one first arc part and at least one first flat part connected to the at least one first arc part, the sliding through hole structure comprises at least one second arc part and at least one second flat part connected to the at least one second arc part, and the at least one second arc part and the at least one second flat part are respectively corresponding to the at least one first arc part and the at least one first flat part.

7. The driving mechanism of claim 6, wherein an internal thread structure is formed on an inner periphery of the third transmission component, and an outer thread structure is formed on the at least one first arc part of the sliding component.

8. The driving mechanism of claim 1, wherein the bracket further comprises a holding portion, the holding portion comprises a rotation through hole structure, and the third transmission component rotatably passes through the rotation through hole structure.

9. The driving mechanism of claim 1, wherein the bracket further comprises a supporting portion, the supporting portion comprises a platform structure, and the platform structure is configured to support a side of the third transmission component.

10. The driving mechanism of claim 1, wherein the bracket further comprises an accommodating portion, the accommodating portion comprises an L-shaped structure, and the L-shaped structure is configured to accommodate the first transmission component.

11. An auto-injector comprising:
a reservoir;
a plunger slidably disposed inside the reservoir; and
a driving mechanism for driving the plunger to slide relative to the reservoir, the driving mechanism comprising:
a first transmission component;
a driving component coupled to the first transmission component and for driving the first transmission component to rotate;
a second transmission component rotatably engaged with the first transmission component, the second transmission component being driven by the first transmission component to rotate when the driving component drives the first transmission component to rotate;
a third transmission component fixedly connected to the second transmission component, the third transmission component being driven by the second transmission component to rotate when the first transmission component drives the second transmission component to rotate;
a sliding component at least partially slidably disposed inside the third transmission component and coupled to the third transmission component, the sliding component being connected to the plunger, the sliding component being driven by the third transmission component to slide relative to the third transmission component when the third transmission component rotates; and
a bracket comprising a guiding portion, the sliding component passing through the guiding portion, the third transmission component being rotatable relative to the bracket, and the guiding portion being configured to guide the sliding component to slide without a rotation.

12. The auto-injector of claim 11, wherein when the driving component drives the first transmission component to rotate around a first rotating axis, the first transmission component drives the second transmission component to rotate around a second rotating axis so as to drive the third transmission component to rotate around the second rotating axis together with the second transmission component, so that the sliding component is driven to slide relative to the third transmission component along a sliding direction parallel to the second rotating axis without the rotation around the second rotating axis.

13. The auto-injector of claim 11, the first transmission component is a worm screw, the second transmission component is a worm gear, the third transmission component is a screw sleeve, the sliding component is a screw rod, and the driving component is an electric motor.

14. The auto-injector of claim 11, wherein the driving mechanism further comprises a reducer coupled between the driving component and the first transmission component.

15. The auto-injector of claim 14, wherein the reducer is a gearbox.

16. The auto-injector of claim 11, wherein the guiding portion comprises a sliding through hole structure, the sliding component slidably passes through the sliding through hole structure, a cross section of the sliding component matches with a cross section of the sliding through hole structure, the sliding component comprises at least one first arc part and at least one first flat part connected to the at least one first arc part, the sliding through hole structure comprises at least one second arc part and at least one second flat part connected to the at least one second arc part, and the at least one second arc part and the at least one second flat part are respectively corresponding to the at least one first arc part and the at least one first flat part.

17. The auto-injector of claim 16, wherein an internal thread structure is formed on an inner periphery of the third transmission component, and an outer thread structure is formed on the at least one first arc part of the sliding component.

18. The auto-injector of claim 11, wherein the bracket further comprises a holding portion, the holding portion comprises a rotation through hole structure, and the third transmission component rotatably passes through the rotation through hole structure.

19. The auto-injector of claim 11, wherein the bracket further comprises a supporting portion, the supporting portion comprises a platform structure, and the platform structure is configured to support a side of the third transmission component.

20. The auto-injector of claim 11, wherein the bracket further comprises an accommodating portion, the accommodating portion comprises an L-shaped structure, and the L-shaped structure is configured to accommodate the first transmission component.
